(19) United States
(12) Patent Application Publication
(10) Pub. No.: US 2008/0124319 A1
(43) Pub. Date: May 29, 2008

(54) Methods for treating inflammation by disrupting MCH-mediated signaling

(76) Inventors: Charalabos Pothoulakis, Waban, MA (US); Efi Kokkotou, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/804,013

(22) Filed: May 16, 2007

Related U.S. Application Data
(60) Provisional application No. 60/800,593, filed on May 16, 2006.

(51) Int. Cl. A61K 39/395; A61P 29/00

(52) U.S. Cl. 424/130.1

(57) ABSTRACT

Disclosed herein are methods for treating an inflammatory condition in a patient comprising administering an agent that inhibits the signaling activity of MCH, thereby inhibiting the inflammatory response in the tissue, and in a mammal comprising administering to the mammal an effective amount of an agent that inhibits MCH activity, MCH binding to an MCH receptor or the signaling activity of an MCH receptor that mediates intestinal inflammation.

### RELATED APPLICATION

This application claims the benefit of U.S. Provisional Application No. 60/800,593, filed on May 16, 2006. The entire teachings of the above application are incorporated herein by reference.

### BACKGROUND OF THE INVENTION

Melanin Concentrating Hormone (MCH) is a hypothalamic neuropeptide that regulates appetite and energy balance. In humans, two types of receptors for MCH have been identified, Melanin Concentrating Hormone Receptor 1 (MCHR1, also known as SLC1 or GPR24) and Melanin Concentrating Hormone Receptor 2 (MCHR2). Apart from the brain, which represents the main target tissue for MCH, MCH receptors are also expressed in various organs, suggesting that MCH may have different physiologic and pathophysiologic effects in the periphery. MCHR1, for example, is also expressed in the adipose tissue, thyroid, kidney, tongue, lung and peripheral blood mononuclear cells (PBMCs). It is unclear if MCH acts as a hormone since only autocrine/paracrine action in response to MCH has been demonstrated. The MCH receptor belongs to the family of seven transmembrane G-protein coupled receptors, and its activation upon ligand binding results in Erk1/2 phosphorylation and lowering of cAMP intracellular levels.

Animals lacking either MCH itself or its receptor MCHR1, are lean, hypophagic and hypermetabolic. Furthermore, mice lacking MCH, when placed on a high fat diet, fail to up-regulate inflammatory markers such as TNFa, MCP1, STATs and SOCS3, and/or to activate the NF-κβ pathway in their white adipose tissue and liver. This effect was initially attributed to the lack of obesity in the MCH deficient mice, but it could also be explained by proinflammatory MCH-associated responses. Prior to the present disclosure, nothing had been known of a putative proinflammatory role exerted by MCH in animals or humans.

### SUMMARY OF THE INVENTION

Previous studies have shown that neuropeptides such as neurotensin, Cortiotropin Releasing Hormone (CRH) and substance P act as proinflammatory cytokines in the gastrointestinal system. Furthermore, leptin, which, like Melanin Concentrating Hormone (MCH), regulates food intake and energy balance, has proinflammatory effects in the gut. Based on these findings, the role of MCH in the pathophysiology of intestinal inflammation was investigated. In the studies described below, for the first time the role of MCH in inflammation induced by the enterotoxin or toxin A from Clostridium difficile (C. difficile), the causative agent of antibiotic associated colitis in animals and humans, was examined.

Disclosed herein is the unexpected and useful discovery that MCH and its receptor type I (MCHR1) are present in the intestine and that they promote intestinal inflammation in an animal model of C. difficile toxin A-mediated enteritis. C. difficile is the primary cause of antibiotic-associated diarrhea and colitis in humans and animals. Mice genetically lacking the MCH receptor have substantially reduced intestinal inflammation following ileal injection of C. difficile toxin A as compared to normal mice. The levels of MCH and its receptor are upregulated in the gut during C. difficile toxin A-mediated enteritis. Injection of MCH or MCH receptor neutralizing antibodies reduces the inflammatory response in the gut, associated with C. difficile toxin A. Since it is known that intestinal damage in response to C. difficile toxin A is mediated via release of proinflammatory cytokines, these results show that MCH, via its intestinal receptor, plays a proinflammatory role in intestinal inflammation. These findings indicate that MCH acts via its colonic receptor to promote colonic inflammation in patients with intestinal inflammation (e.g., inflammatory bowel disease, diarrhea, Crohn's disease and ulcerative colitis). These findings allow for the treatment of patients with acute and chronic enterocolitis either from bacterial, viral, or toxin mediated etiology (e.g., inflammatory bowel disease, diarrhea, Crohn's disease and ulcerative colitis). Additionally, these data suggest treatment of patients with other inflammatory and/or autoimmune disorders in tissues where MCHR1 is present, such as, for example, thyroid, kidney, skin and blood cells.

In a preferred embodiment, the invention is directed to a method for treating an inflammatory condition in a patient comprising administering an agent that inhibits the signaling activity of MCH, thereby inhibiting the inflammatory response in the tissue.

In a particular embodiment, the agent is selected from the group consisting of: an MCH antagonist, an MCH antibody or antigen-binding fragment thereof, an MCH derivative, an MCH inhibitor, an MCH receptor peptide or fragment, an MCH receptor inhibitor, an MCH receptor antagonist, an MCH receptor antibody or antigen-binding fragment thereof, an MCH analog, and combinations thereof. In one embodiment, the inflammatory condition is mediated by a bacterium, a virus or a toxin. In one embodiment, the toxin is produced by C. difficile, such as, for example, C. difficile toxin A. In one embodiment, the inflammatory condition is selected from the group consisting of: acute and chronic enterocolitis, ulcerative colitis, inflammatory bowel disease and autoimmune disorders in tissues where MCHR1 is expressed. In a particular embodiment, the inflammatory bowel disease is Crohn's disease. In one embodiment, the autoimmune disorders occur in a tissue selected from the group consisting of: thyroid, kidney, skin and blood cells.

In another embodiment, the invention is directed to a method for preventing upregulation of one or more inflammatory markers in a cell comprising inhibiting MCH. In a particular embodiment, the inflammatory marker is selected from the group consisting of: TNFa, MCP1, STAT markers and SOCS3.

In another embodiment, the invention is directed to a method for inhibiting activation of the NF-κβ pathway in white adipose tissue and liver comprising inhibiting MCH.

In another embodiment, the invention is directed to a method of treating inflammatory diarrhea in a mammal comprising administering to the mammal an effective amount of an agent that inhibits MCH activity, MCH binding to an MCH receptor or the signaling activity of an MCH receptor that mediates intestinal inflammation.

In another embodiment, the invention is directed to a method of inhibiting inflammatory damage to a cell comprising inhibiting the signaling activity of MCH.

In yet another embodiment, the invention is directed to a method of treating C. difficile toxin A-mediated enteritis comprising inhibiting the signaling activity of MCH.

### BRIEF DESCRIPTION OF THE DRAWINGS

FIGS. 1A and 1B are graphical representations of data depicting protection from toxin A induced ileitis in mice. Wild-type (WT) and MCH-deficient mice (n=4-5 per group) were anesthetized, and mouse ileal loops were injected with toxin A. After four hours, mice were sacrificed and ileal loops were harvested for histological examination and gene expression analysis. FIG. 1A: Quantitation of microscopic damage due to toxin A treatment in ileal loops of WT and MCH knockout (MCH-KO) mice was evaluated as the sum of the score of three different histological parameters for each mouse. The mean values +/−SE of each group are presented (*p<0.05 between WT and MCH-KO toxin A treated mice). FIG. 1B: RNA was prepared from toxin A treated ileal loops and gene expression levels of TNFα, IFNγ, ILNβ and IL-4 were assessed by real time RT-PCR. Results are expressed as the mean +/−SE for each group. Expression of all cytokines was found to be significantly (p<0.05) decreased in the group of MCH-KO mice.

FIG. 2 shows images of murine intestinal histological sections showing MCH and MCHR1 immunoreactivity. Histological sections of intestinal tissue from WT mice were stained for MCH (left upper panel) or MCHR1 (left lower panel) using a rabbit polyclonal antibody followed by a fluorochrome labeled secondary antibody. In the right panels, the staining of sequential sections is presented where the primary antibody has been omitted (negative control). MCH immunostaining (left upper panel) is localized in the intestinal mucosa as well as in the muscularis. MCHR1 is (lower left panel) also abundantly expressed in the intestinal mucosa, as well as the muscularis, with strong signal expressed in intestinal epithelial cells and cells of the intestinal lamina propria. Very little non-specific staining is present in tissues where the primary antibodies for MCH (right upper panel) or the MCHR1 (right lower panel) were omitted (magnification 40×).

FIGS. 3A and 3B are a graphical representation and Western blot, respectively, showing upregulation of MCHR1 mRNA and protein levels in mouse intestine exposed to toxin A. Ileal loops of WT mice were injected with either buffer (control) or toxin A for 30 minutes, 2 hours or 4 hours (n=6/time point) and tissues were harvested for measurements of either MCHR1 mRNA (FIG. 1A) by real time PCR (results expressed as mean +/−SEM) or protein (FIG. 1B) by Western blot analysis (a representative experiment is shown). FIG. 1B: lanes 1-3: controls; lanes 4 and 5: toxin A exposure for 30 min, lanes 6 and 7: toxin A exposure for 2 hours, lanes 8 and 9: toxin A exposure for 4 hours (p<0.05 for comparisons among toxin A treated and buffer treated groups).

FIGS. 4A and 4B are graphical representations showing the effect of MCH or MCHR1 neutralization on toxin A-induced ileitis. Mice (n=6/group) were treated twice (−12 hours and −2 hours) with 1 mg/kg of MCH, MCHR1 or control antiserum. Ileal loops were subsequently injected with toxin A. After four hours of treatment, histological scoring of inflammation (FIG. 4A) an intestinal fluid secretion (FIG. 4B) and proinflammatory cytokines mRNA expression were evaluated. Results are expressed as mean +/−SE (p<0.05 for all comparisons among control antibody and specific antibody treatments).

### DETAILED DESCRIPTION OF THE INVENTION

The present invention is based on the novel and unexpected discovery that mice lacking the gene for MCH have reduced inflammatory response following ileal administration of purified toxin A. Furthermore, immunohistochemical as well RNA expression studies in wild-type (WT) mice showed the presence of MCH- and MCHR1-positive cells in buffer-exposed ileum. This expression was found by Immunoblot as well as Quantitative RT-PCR to be increased after toxin A exposure in WT mice. Treatment of WT mice with MCH or MCH receptor neutralizing antibodies resulted in reduced toxin A-associated pathology, secretion of fluid and intestinal inflammation. It was concluded from these studies that MCH participates in the pathophysiology of toxin A-induced intestinal inflammation, a condition associated with upregulation of MCH as well as its MCH receptor in the small intestine and colon. This is the first demonstration of the presence and a functional role of MCH and MCHR1 in the pathophysiology of enterotoxin-mediated secretion and inflammation or any other form of inflammation in the gut. Along these lines, MCH mRNA levels are also increased in the colon of animals injected with TNBS, an animal model of inflammatory bowel disease. Thus, MCH antagonists or MCH/MCHR1 immunotherapy are shown to be novel therapeutic modalities in gastrointestinal inflammatory diseases.

The present invention therefore includes a method of treating MCH-mediated intestinal inflammation, comprising inhibiting or decreasing MCH activity, MCH binding to its receptor, or the signaling activity of MCHR (e.g., MCHR1). Such a treatment can be accomplished by administration of an agent. An agent can be any molecule, chemical or biological, that modulates the activity of MCH or the MCH receptor. Administering the agent can be accomplished by directly contacting MCH receptor positive cells with the agent, or by delivery to MCH receptor positive cells of the agent in a composition with a pharmacologically or physiologically acceptable carrier. Methods are known in the art to contact or deliver an agent to a target tissue or tissue-specific cells (e.g., epithelial and lamina propria cells).

The invention encompasses modulation of MCH activity or MCH receptor activity in vertebrates, and, more specifically, mammals. The methods and of the present invention are suitable for veterinary use as well as for treating humans. For example, canines exposed to toxins that result in MCH-mediated intestinal inflammation can be treated using the methods and/or agents described herein.

MCH-mediated inflammation occurs in intestinal tissues (e.g., the small or large intestine, ileum or colon). This inflammation is characterized by fluid secretion, diarrhea and elevated cytokine levels. The inflammation can be mediated by a bacteria (e.g., Clostridium difficile), a virus or a toxin. Such a toxin can be produced by a bacterium (e.g., TxA produced by C. difficile). Alternatively, the inflammation can be mediated by an autoimmune response (Chan, J. et al., In press. Diabetes). The intestinal inflammation can be that caused by any inflammatory response such as, for example, a parasitic infection, autoimmune inflammation, a response associated with a disease, such as inflammatory bowel disease (IBD), Crohn's disease, ulcerative colitis, acute enterocolitis or chronic enterocolitis.

An agent “modulates” activity if it alters the activity from that which would be exhibited in the absence of the agent. For example, inhibitors decrease activity, e.g., functional inhibitors that interact and block an active site, or competitive inhibitors that compete for binding; antagonists inhibit binding activity, e.g., molecules that reduce binding affinity between a receptor and ligand; and agonists increase binding activity, e.g., molecules that increase binding affinity between a receptor and a ligand. Examples of such molecules include, but are not limited to, MCH antibodies, small molecule agents, MCH agonists, MCH antagonists, non-biologically active MCH analogs, soluble MCH receptors, MCH receptor agonists or MCH receptor antagonists. These agents can be proteins, peptides, peptide analogs, or chemical compounds or derivatives.

The invention encompasses agents that are antibodies and antisera that can be used for inhibiting the activity of MCH and the binding of MCH to its receptor, thereby mitigating the intestinal inflammation. These antibodies can specifically bind to the MCH receptor located on intestinal cells, thus preventing MCH binding to the receptor, and, thereby, inhibiting or decreasing MCH receptor signaling and the resulting MCH-mediated inflammatory response. Such antibodies and antisera can be combined with pharmaceutically-acceptable compositions and carriers to form compositions. The antibodies can be either polyclonal antibodies or monoclonal antibodies.

MCH, the MCH receptor, or antigenic epitopes of MCH or the MCH receptor can be used to generate antibodies that are specific for MCH or its receptor. For use as an antigen, MCH or the MCH receptor can be recombinantly produced or engineered as described in, e.g., WO 96/05309; U.S. Pat. No. 5,552,522; U.S. Pat. No. 5,552,523; and U.S. Pat. No. 5,552,524, the teachings of which are incorporated by reference. MCH or the MCH receptor can also be produced by chemical synthesis, or isolated from mammalian plasma using methods well-known to those of skill in the art. For example, MCH used to induce antibody production can be intact protein, e.g., the full-length polypeptide (Zhang, Y. et al., 1994. Nature, 372:425-432).

Specifically included in the present invention are agents that are MCH analogs or derivatives of either MCH or the MCH receptor. Analogs, as used herein, are molecules that are structurally similar to, for example, MCH, and act to compete with MCH for MCH receptor binding sites. MCH or MCH receptor or derivatives, as used herein, are peptides or proteins having amino acid sequences analogous to endogenous MCH or the MCH receptor. MCH derivatives can be used, for example, as a competitive inhibitor of MCH binding by competing for MCH receptor binding sites. The present invention includes the use of such MCH derivatives that are able to bind to the MCH receptor, but do not induce the MCH-mediated inflammatory response. MCH receptor derivatives can be used, for example, to sequester unbound MCH, thereby reducing the MCH levels available to bind and induce endogenous MCH receptors. Analogous amino acid sequences are defined herein to mean amino acid sequences with sufficient identity of amino acid sequence of endogenous MCH to possess the biological activity of endogenous MCH or a slightly altered activity, e.g., reduced MCH receptor binding affinity, as well as analogous proteins that exhibit greater, or lesser activity than endogenous MCH. The derivatives or analogs of the present invention can also be “peptide mimetics,” peptides or proteins that contain chemically modified or non-naturally occurring amino acids. These mimetics can be designed and produced by techniques known to those of skill in the art (see, e.g., U.S. Pat. Nos. 4,612,132; 5,643,873 and 5,654,276, the teachings of which are herein incorporated by reference).

The present invention also encompasses the administration of fusion proteins comprising MCH, MCH receptor, or derivatives thereof, referred to as a first moiety, linked to a second moiety not occurring in the MCH or MCH receptor protein. The second moiety can be a single amino acid, peptide or polypeptide or other organic moiety, such as a carbohydrate, a lipid, or an inorganic molecule. Examples of a second moiety include, for example, maltose or glutathione-S-transferase. The second moiety can also be a targeting moiety used to target the fusion protein to intestinal tissue.

Where the MCH receptor is membrane-bound, the present invention also provides for inhibiting MCH signaling using soluble isoforms of OB-R, e.g., Ob-Re (Takaya, K. et al., 1996. Biochem. Biophys. Res. Commun., 225:75-83) and engineered soluble forms of the MCH receptor. These soluble forms of the MCH receptor would act to bind to unbound MCH, thereby sequestering MCH free in solution and preventing binding of the free MCH to membrane-bound MCH receptor. For these MCH-receptor isoforms and derivatives, part or all of the intracellular and transmembrane domains of the protein are deleted such that the protein is fully secreted from the cell in which it is expressed. The intracellular and transmembrane domains of the MCH receptor can be identified in accordance with known techniques for determination of such domains from sequence information. Commercially and freely available software, such as TopPred2 (Stockholm, Sweden), can be used to predict the location of transmembrane domains in an amino acid sequence, domains which are described by the location of the center of the transmembrane domain, with at least ten transmembrane amino acids on each side of the reported central residue(s).

Systematic substitution of amino acids within the MCH protein can also be used to engineer high-affinity protein agonists and antagonists to the MCH receptor. Accordingly, the engineered MCH would exhibit enhanced or diminished affinity for binding with the MCH receptor. Such agonists and antagonists can be used to suppress or modulate the activity of MCH, thereby mitigating diarrhea or intestinal inflammation. Antagonists to MCH are applied in situations of gut inflammation, to block the inhibitory effects of MCH and mitigate the inflammation.

Candidate MCH receptor inhibitors or antagonists can also be identified by evaluating the binding of MCH to its receptor in the presence and absence of the candidate inhibitor antagonist. Such techniques are well-known to those of skill in the art. Alternatively, candidate MCH receptor inhibitors or antagonists can be identified by measuring MCH receptor signaling activity by the methods described herein (e.g., measurement of fluid secretion).

Administering agents of the present invention can be accomplished either by administering the agent alone (naked administration) or by administering the agent as part of a composition. Modes of administering the agents or compositions of the present inventions include aerosol, ingestation, intravenous, intramuscular, intraperitoneal, intrasternal, subcutaneous and intraarticular injection and infusion.

The formulations include those suitable for oral, rectal, nasal, topical (including buccal and sublingual), intrauterine, vaginal or parenteral (including subcutaneous, intraperitoneal, intramuscular, intravenous, intradermal, and epidural) administration. The formulations may conveniently be presented in unit dosage of therapeutically effective amounts and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

The compositions containing inhibitors of MCH or the MCH receptor may also contain other proteins or chemical compounds. The composition may further contain other agents which either enhance the activity of the inhibitor or compliment its activity or use in treatment. Such additional factors and/or agents may be included in the composition to produce a synergistic effect with the inhibitor of MCH or the MCH receptor, or to minimize side effects. Pharmaceutical or physiological compositions for parenteral injection comprise pharmaceutically or physiologically acceptable, herein used interchangeably, sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by trapping the drug in liposomes or microemulsions that are compatible with body tissues. Additionally, administration of the inhibitor of MCH or the MCH receptor of the present invention may be administered concurrently with other therapies.

Alternatively, it may be undesirable to administer the protein systemically because of side-affects. To eliminate pleiotropic effects of administering an agent included in the present invention, it would be useful to deliver (or target) the agent to a specific tissue (e.g., intestinal tissue or MCH receptor positive epithelial or lamina propria cells). One way to deliver the agent to a specific tissue is to conjugate the protein with a targeting agent. For example, the protein can comprise a peptide to target the MCH receptor to a specific tissue or cell type, e.g., intestinal tissue or cells. Such targeting molecules are well known to those of skill in the art.

Agents can be used in compositions with carriers known in the art. Such carriers can be used as vehicles that target specific tissues or cell types (e.g., intestinal tissue or MCH receptor positive epithelial or lamina propria cells), are they can be used to increase the stability or efficacy of the agent. Such a composition can also contain (in addition to inhibitor and a carrier) diluents, fillers, salts, buffers, stabilizers, solubilizers, and other materials well known in the art. The term “pharmaceutically acceptable” can be used interchangeably with “physiologically acceptable” to mean a non-toxic material that does not interfere with the effectiveness of the biological activity of the active ingredient(s). The characteristics of the carrier will depend on the route of administration. In addition, an agent, e.g., inhibitor of MCH or the MCH receptor, may be active as a monomer or multimer (e.g., heterodimers or homodimers) or may complex with itself or other proteins or molecules. As a result, compositions of the invention may comprise an agent in such multimeric or complexed form. Such multimers, or complexes, are especially useful, for example, to prolong the half-life of the protein in circulation.

The agents of the present invention can be in the form of a liposome in which the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

The compositions can be administered intravenously, as by injection of a unit dose, for example. The term “unit dose” is an effective amount of the agent that, when used in reference to a composition of the present invention, refers to physically discrete units suitable as unitary dosage for the subject, each unit containing a predetermined quantity of active material calculated to produce the desired effect in association with the required diluent, i.e., carrier or vehicle. As used herein, an effective amount of an agent is that determined by one of ordinary skill in to be the amount necessary to decrease or completely inhibit the inflammatory response mediated by MCH and the MCH receptor in a specific tissue or cell. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions that may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

When an effective amount of the inhibitor of MCH or the MCH receptor of the present invention is administered orally, the composition of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% inhibitor of the present invention, and preferably from about 25 to 90% inhibitor of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of inhibitor of the present invention, and preferably from about 1 to 50% inhibitor of the present invention.

When an effective amount of the inhibitor of MCH or the MCH receptor of the present invention is administered by intravenous, cutaneous or subcutaneous injection, inhibitor of the present invention will be in the form of a pyrogen-free, parenterally acceptable aqueous solution. The preparation of such parenterally acceptable inhibitor solutions, having due regard to pH, isotonicity, stability, and the like, is within the skill in the art. A preferred pharmaceutical composition for intravenous, cutaneous, or subcutaneous injection should contain, in addition to the inhibitor or agonist of the present invention, an isotonic vehicle such as sodium chloride, Ringer's solution, dextrose, dextrose and sodium chloride, lactated Ringer's solution, or other vehicles known in the art. The pharmaceutical composition of the present invention may also contain stabilizers, preservatives, buffers, antioxidants, or other additives known to those of skill in the art.

By “contacting” is meant not only topical application, but also those modes of delivery that introduce the composition into the tissues, or into the cells of the tissues (e.g., intestinal tissue or MCH receptor positive epithelial or lamina propria cells).

Use of timed release or sustained release delivery systems are also included in the invention. Such systems are highly desirable in situations where surgery is difficult or impossible, e.g., patients debilitated by age or the disease course itself, or where the risk-benefit analysis dictates control over cure.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

Additionally, osmotic minipumps may also be used to provide controlled delivery of high concentrations of inhibitor or agonist of MCH or the MCH receptor through cannulae to the site of interest (e.g., delivery of the inhibitor specifically to, for example, intestinal tissue or MCH receptor positive epithelial or lamina propria cells). The biodegradable polymers and their use are known to those of skill in the art, for example, as detailed in Brem et al. (1991. J. Neurosurg. 74:441-446), which is hereby incorporated by reference in its entirety.

The methods of the present invention contemplate single as well as multiple administrations, given either simultaneously or over an extended period of time. In addition, agents suitable for use in the present invention can be administered in conjunction with other forms of therapy, e.g., immunotherapy. The duration of intravenous therapy using the pharmaceutical composition of the present invention will vary depending on the severity of the disease being treated and the condition and potential idiosyncratic response of each individual recipient. It is contemplated that the duration of each application of the inhibitor of the present invention will be in the range of 12 to 24 hours of continuous intravenous administration. Ultimately the attending physician will decide on the appropriate duration of intravenous therapy using the pharmaceutical composition of the present invention.

Preferred unit dosage formulations are those containing a daily dose or unit, daily sub-dose, or an appropriate fraction thereof, of the administered ingredient. It should be understood that in addition to the ingredients, particularly mentioned above, the formulations of the present invention may include other agents conventional in the art having regard to the type of formulation in question.

This invention is illustrated further by the following examples, which are not to be construed as limiting in any way.

### EXEMPLIFICATION

### Example 1

### Reduced Toxin A-Induced Inflammation in MCH-Deficient Mice

Twelve weeks old male C57B16 wild-type (+/+) and MCH-deficient (−/−) mice weighting 20-25 g were housed under controlled conditions on a 12-12 h light dark cycle. Mice were fasted for 16 hours before the experiments to avoid formation of stool, but had free access to a 5% sucrose solution to prevent hypoglycemia and hypothermia. Mice were anesthetized with a mixture of ketamine (0.9 mL) and xylazine (0.1 mL) in 9 mL of sterile water at a dose of 0.15 mL/20 g body weight. A laparotomy was performed and a 2-3 cm long loop was formed at the terminal ileum as previously described (Pothoulakis, C. et al., 1994. Proc. Natl. Acad. Sci. USA, 91:947-51; Castagliuolo, I. et al., 1999. J. Clin. Invest., 103:843-9). Loops were injected with either 0.15 mL of phosphate buffer saline (PBS) (pH 7.4) containing 10 g of purified toxin A or buffer alone (control). The abdomen was then closed and animals were placed on a heating pad at 37° C. for the duration of the experiment. At the end of treatment, animals were sacrificed with CO₂ inhalation and intestinal loops were removed and preserved for histology or RNA extraction.

### Histological Assessment of Inflammation

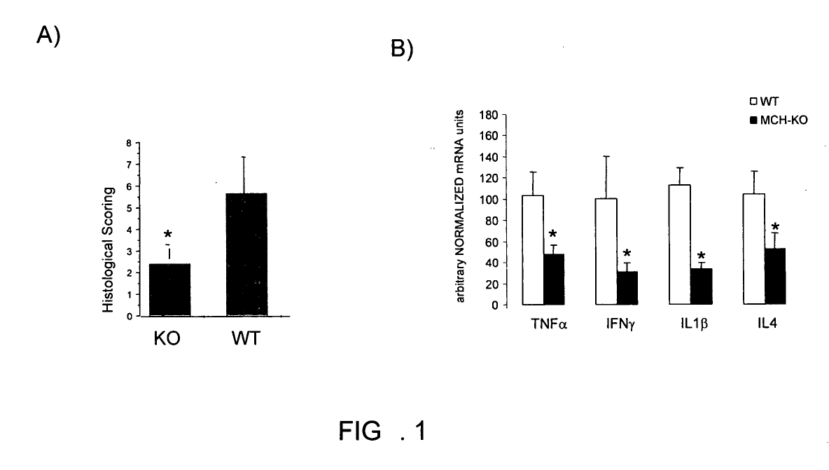

Microscopic Damage Scores: Transverse sections (5 μm thick) of the ileal loops were fixed in formalin, paraffin-embedded, and stained with hematoxylin and eosin. Histologic severity of enteritis was graded by a “blinded” gastrointestinal pathologist using previously established toxin A-associated histologic parameters. Three different parameters (epithelial cell damage, congestion and edema, and neutrophil infiltration) were scored on a scale from 0 to 3. Scores for each group were then added and averaged, and results were expressed as mean +/−SEM. As shown in FIG. 1A, MCH knockout (MCH-KO) mice exhibited significantly less microscopic damage in response to toxin A administration compared to the wild-type mice (p<0.05). This result strongly suggests that MCH plays a significant role in the pathogenesis of C. difficile toxin A-associated histologic damage and inflammation.

### Molecular Assessment of Inflammation

Intestinal inflammatory conditions are characterized by the increased expression of a cascade of inflammatory cytokines such as TNFα, IFNγ, IL1β and IL-4. Induction of these cytokines in the mouse ileal loops that were exposed to toxin A was assessed at their mRNA level using Real-Time Q-PCR. The effect of toxin A treatment in wild-type (WT) versus MCH-KO mice was assessed. Total RNA was isolated from ileal loops using the RNeasy mini kit (Qiagen, Valencia, Calif.). Fifty nanograms of RNA were subjected to RT-PCR using the TaqMan One Step RT-PCR reagents, gene-specific primers and FAM labeled probe (Applied Biosystems, Foster City, Calif.). The samples were run in duplicate and the values obtained were normalized by GAPDH expression. As shown in FIG. 1B, following ileal toxin A exposure MCH-KO mice exhibited 2-3 times lower TNFα, IFNγ, IL10 and IL-4 mRNA levels than the WT mice (p<0.05).

### Presence of Immunoreactive MCH and MCHR1 in the Intestinal Epithelium

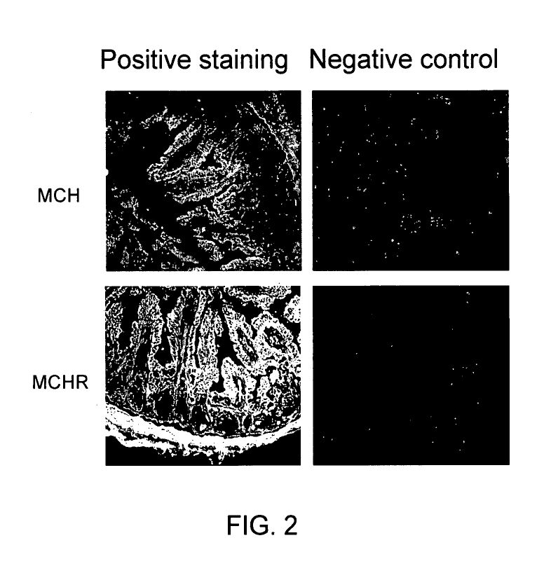

Transverse frozen sections (5 μm thick) of mouse ileal tissue were stained for the presence of MCH or MCHR1. For MCH immunostaining a rabbit polyclonal antibody was used (Ludwig, D. et al., 2001. J. Clin. Invest., 107:379-86). MCH peptide sequences are identical in mouse, human and rat; thus the antibody recognizes MCH from all three species. The MCHR1 antibody was developed in rabbits (BioSource International, Camarillo, Calif.) against the peptide, ASQRSIRLRTKRVTR (SEQ ID NO: 1). The antibody recognizes both the human and the mouse MCHR1. The sections were fixed in cold 80% acetone, air dried, and washed in TBS. They were pre-incubated (1 hr, RT) in TBS with normal goat serum (5%), drained, then incubated (2 hr., RT) with the primary antibody (1:5000 dilution). The sections were then washed in TBS and incubated with goat anti-rabbit FITC-conjugate (Jackson ImmunoResearch Laboratories, West Grove, Pa.; 1 hr., RT, 1:100 dilution), washed, and coverslipped with Vectashield anti-fade medium (Vector Laboratories, Burlingame, Calif.). Images were viewed under a fluorescent microscope and representative results are shown in FIG. 2. The right panel represents non-specific staining at adjacent sections, where the primary antibody for either MCH or MCHR1 had been omitted. MCH immunostaining (FIG. 2, upper left panel) is localized in the intestinal mucosa as well as in the muscularis. MCHR1 (FIG. 2, lower left panel) is also abundantly expressed in the intestinal mucosa, as well as the muscularis, with strong signal expressed in intestinal epithelial cells and cells of the intestinal lamina propria. Very little staining is present in tissues where the primary antibodies for either MCH (FIG. 2, upper right panel) or MCHR1 (FIG. 2, lower right panel) were omitted. These results indicate that MCH and its receptor are expressed abundantly in mouse ileum.

### Toxin A Increases Intestinal MCH Receptor mRNA Levels in Normal Mice

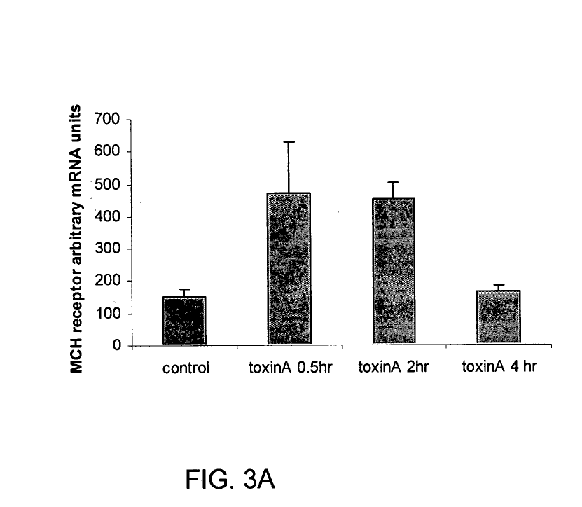

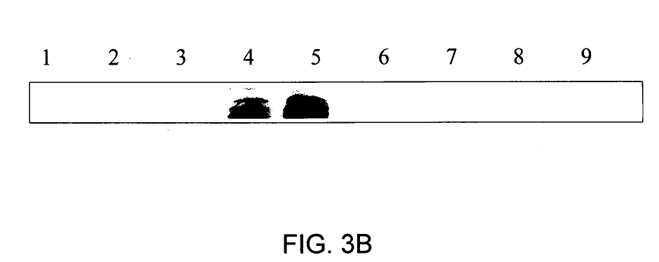

In a similar to above described experiment, RNA was prepared form ileal loops of WT mice treated with toxin A for 30 min, 2 hrs or 4 hrs, or with buffer for 4 hrs. Ten nanograms of RNA was subsequently analyzed by Real-Time PCR using previously described primers and probes (Kokkotou, E. et al., 2001. Endocrinology, 142:680-6). Toxin A treatment of mouse ileal loops resulted in a significant 3.5-fold increase of MCHR1 mRNA levels between 30 mins and 2 hrs of treatment (FIG. 3A).

### Increased MCH Receptor Immunoreactivity following Toxin A Administration in Mouse Ileum

Toxin A or buffer was injected into loops of terminal ileum (see above for Methods) of 12 week old male CD1 mice (n=6 per group). After 30 min, 2 hr and 4 hr following toxin A exposure, and 4 hr following buffer exposure, animals were sacrificed and ileal loops were removed and homogenized in lysis buffer at a concentration of 1 mg of tissue/mL of lysis buffer. 30 mg of protein were then subjected to electrophoresis in a 10% Tris-Glycine gel (MCHR1) and transferred to an Immobilon-P membrane (Millipore, Bedford, Mass.). The blot was incubated for 1 hr at RT with the anti-MCH receptor primary antibody described above, at a dilution of 1:1000, followed by an incubation with a secondary HRP-labeled goat anti-rabbit antibody at a dilution of 1:3000. Visualization of proteins was achieved by using the Supersignal West Pico Chemiluminescent Substrate (Pierce, Rockford, Ill.). FIG. 3B depicts results of a representative experiment. There was no detectable signal for MCH receptor 1 protein in control, buffer injected intestine. MCH receptor is significantly up-regulated 30 min after injection of toxin A into ileal loops, however.

### Treatment with Antibodies Against MCH or MCHR1 Prevent Toxin A Induced Enteritis

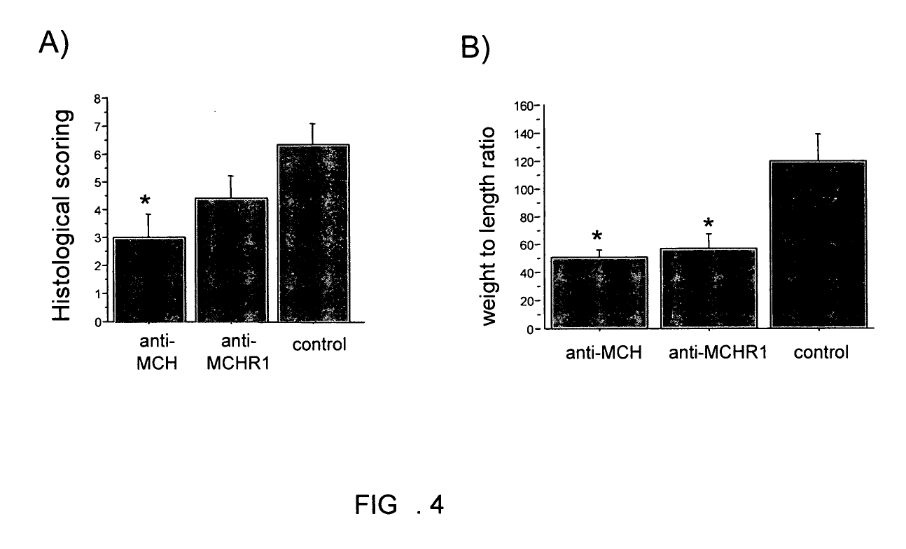

Having demonstrated that mice lacking functional MCH are protected from toxin A-mediated intestinal inflammation, an examination was undertaken to determine if neutralization of MCH or MCHR1 by use of specific antibodies could mimic this effect. Wild-type 12 week old male mice (n=8 per group) were treated IP at 12 hr and 2 hr prior to toxin A exposure with 1 mg/kg with the antibodies described above or control antibody (preimmune serum). Four hours after toxin A injection in mouse ileal loops, tissue was harvested and processed for histology or RNA preparation. As shown in FIG. 4A, the microscopic damage score in mice that received the anti-MCH antibody was reduced by 52% compared to mice that received the control antibody. Likewise, mice that received the anti-MCHR1 antibody had a histological damage score that was 70% of that of the control mice. This protective effect of the anti-MCH/MCHR1 antibodies is also reflected by the amount of fluid secretion within the ileal loops, described by the weight to length ratio of the ileal loops, another marker for the severity of toxin A-induced inflammation (Pothoulakis C. et al., 1994. Proc. Natl. Acad. Sci. USA, 91:947-51; Sartor, R. 1994. Gastroenterology, 106:533-9). As shown in FIG. 4B, mice that received the specific antibodies had a reduced loop weight to length ratio (by about 50%) compared to that of mice receiving control antibody (p<0.001). Thus, an antibody directed against MCH or its receptor can reduce fluid secretion and histologic responses associated to C. difficile toxin A-induced inflammation in mouse intestine.

### MCH is Up Regulated in the Acute Phase of TNBS Colitis

Ulcerative colitis (UC) and Crohn's disease (CD) are chronic debilitating inflammatory diseases that exhibit a clinical course characterized by successive exacerbation and remissions. While the causative agent(s) has not been identified, considerable evidence suggests that inflammatory mediators amplify the inflammatory process and produce mucosal dysfunction. An important area in intestinal pathophysiology is how brain-gut hormones and neuropeptides modulate intestinal inflammatory responses. The discovery that MCH is linked to intestinal inflammation led to an examination of MCH mRNA expression in the colon of animals with TNBS colitis, an animal model of Crohn's disease (Vergnolle, N. et al., 1997. Am. J. Physiol., 273:R623-9). For the induction of TNBS colitis in mice, after overnight fasting and anesthesia, a 50 μL enema of 250 mg/kg of 2,4,6-trinitrobenzene sulfonic acid (TNBS) (Fluka, Buchs, Switzerland), or saline in 35% ethanol was infused into the colonic lumen (3.5 cm from the anal verge) via a 1 mL syringe (Becton Dickinson, Franklin Lakes, N.J.) fitted with a polyethylene cannula (Intramedic PE-20 tubing; Becton Dickinson). After the infusion, the mice were maintained in a supine Trendelenberg position until recovery from anesthesia in order to prevent early leakage of the intracolonic instillate. Two days after TNBS treatment, the animals (n=6 per group) were sacrificed and the distal colon was harvested for RNA extraction. MCH mRNA expression levels were evaluated by Real-Time RT-PCR as described above. It was found that MCH mRNA is present in the mouse colon and upregulated (about 4-fold) 48 hr after TNBS treatment (FIG. 5). These results indicate that MCH plays a role in the pathophysiology of colitis.

While this invention has been particularly shown and described with references to preferred embodiments thereof, it will be understood by those skilled in the art that various changes in form and details may be made therein without departing from the scope of the invention encompassed by the appended claims.

1. A method for treating an inflammatory condition in a patient comprising administering an agent that inhibits the signaling activity of MCH, thereby inhibiting the inflammatory response in the tissue.

2. The method of claim 1, wherein the agent is selected from the group consisting of: an MCH antagonist, an MCH antibody or antigen-binding fragment thereof, an MCH derivative, an MCH inhibitor, an MCH receptor peptide or fragment, an MCH receptor inhibitor, an MCH receptor antagonist, an MCH receptor antibody or antigen-binding fragment thereof, an MCH analog, and combinations thereof.

3. The method of claim 1, wherein the inflammatory condition is mediated by a bacterium, a virus or a toxin.

4. (canceled)

5. The method of claim 3, wherein the toxin is C. difficile toxin A.

6. The method of claim 1, wherein the inflammatory condition is selected from the group consisting of: acute and chronic enterocolitis, ulcerative colitis, inflammatory bowel disease and autoimmune disorders in tissues where MCHR1 is expressed.

7. The method of claim 6, wherein the inflammatory bowel disease is Crohn's disease.

8. The method of claim 6, wherein the autoimmune disorders occur in a tissue selected from the group consisting of: thyroid, kidney, skin and blood cells.

9-11. (canceled)

12. A method of treating inflammatory diarrhea in a mammal comprising administering to the mammal an effective amount of an agent that inhibits MCH activity, MCH binding to an MCH receptor or the signaling activity of an MCH receptor that mediates intestinal inflammation.

13. The method of claim 12, wherein the agent is selected from the group consisting of: an MCH antagonist, an MCH antibody or antigen-binding fragment thereof, an MCH derivative, an MCH inhibitor, an MCH receptor peptide or fragment, an MCH receptor inhibitor, an MCH receptor antagonist, an MCH receptor antibody or antigen-binding fragment thereof, an MCH analog, and combinations thereof.

14. The method of claim 12, wherein the inflammatory condition is mediated by a bacterium, a virus or a toxin.

15. (canceled)

16. The method of claim 14, wherein the toxin is C. difficile toxin A.

17. The method of claim 12, wherein the inflammatory condition is selected from the group consisting of: acute and chronic enterocolitis, ulcerative colitis, inflammatory bowel disease and autoimmune disorders in tissues where MCHR1 is expressed.

18. The method of claim 17, wherein the inflammatory bowel disease is Crohn's disease.

19-20. (canceled)

21. A method of treating C. difficile toxin A-mediated enteritis comprising inhibiting the signaling activity of MCH.

22. The method of claim 21, wherein the agent is selected from the group consisting of: an MCH antagonist, an MCH antibody or antigen-binding fragment thereof, an MCH derivative, an MCH inhibitor, an MCH receptor peptide or fragment, an MCH receptor inhibitor, an MCH receptor antagonist, an MCH receptor antibody or antigen-binding fragment thereof, an MCH analog, and combinations thereof.

23. The method of claim 21, wherein the inflammatory condition is mediated by a bacterium, a virus or a toxin.

24. (canceled)

25. The method of claim 23, wherein the toxin is C. difficile toxin A.

26. The method of claim 21, wherein the inflammatory condition is selected from the group consisting of: acute and chronic enterocolitis, ulcerative colitis, inflammatory bowel disease and autoimmune disorders in tissues where MCHR1 is expressed.

27. The method of claim 26, wherein the inflammatory bowel disease is Crohn's disease.

28. (canceled)


SEQUENCE LISTING (1 sequence)

SEQ ID NO: 1 - protein, 15 aa, Artificial Sequence
  synthetic peptide
ASQRSIRLRTKRVTR